(19) United States
(12) Patent Application Publication
(10) Pub. No.: US 2017/0362237 A1
(43) Pub. Date: Dec. 21, 2017

(54) WATER SOLUBLE SALTS OF ALDOSE REDUCTASE INHIBITORS FOR TREATMENT OF DIABETIC COMPLICATIONS

(71) Applicant: Banavara L. Mylari, Lutz, FL (US)

(72) Inventor: Banavara L. Mylari, Lutz, FL (US)

(21) Appl. No.: 15/186,104

(22) Filed: Jun. 17, 2016

(51) Int. Cl. C07D 487/04; C07D 417/06; C07C 279/14; C07C 215/40; C07C 229/26; C07C 229/24; C07H 5/06; C07C 279/26

(52) U.S. Cl. CPC C07D 487/04; C07H 5/06; C07D 417/06; C07C 279/26; C07C 215/40; C07C 229/26; C07C 229/24; C07C 279/14

(57) ABSTRACT

The present invention relates to pharmaceutically acceptable water soluble salts of aldose reductase inhibitors, 2-(8-oxo-7-((5-trifluromethyl)-1H-benzo[d]imidazol-2-yl)methyl)7,8-dihydropyrazin[2,3-d]pyridazin-5-yl)acetic acid and [4-oxo-(5-trifluoromethyl-benzothaiazol-2-yl)methyl)-3,4-dihydro-phthalazin-1-yl]-acetic acid (also known as zopolrestat), pharmaceutical compositions thereof and methods of treating diabetic complications in mammals comprising administering to mammals these salt and compositions.

### CROSS-REFERENCE TO RELATED APPLICATION

This application is a continuation of U.S. patent application Ser. No. 14/177,446, filed on Feb. 11, 2014, which claims priority to U.S. Provisional Application Ser. No. 61/764,001, filed Feb. 13, 2013, the entire contents of which are incorporated by reference.

### FIELD OF THE INVENTION

This invention relates to pharmaceutically acceptable water soluble of salts of aldose reductase inhibitors, and methods of treating diabetic complications in mammals comprising administrating effective amount of one or more such salts to a patient.

### BACKGROUND OF THE INVENTION

The present invention relates to pharmaceutically acceptable water soluble salts aldose reductase inhibitors, 2-(8-oxo-7-((5-trifluoromethyl)-1H-benzo[d]imidazol-2-yl)methyl)7,8-dihydropyrazin[2,3-d]pyridazin-5-yl)acetic acid and [4-oxo-(5-trifluoromethyl-benzothaiazol-2-yl)methyl)-3,4-dihydro-phthalazin-1-yl]-acetic acid (also know as zopolrestat), pharmaceutical compositions thereof and methods of treating diabetic complications in mammals comprising administering to mammals these salt and compositions. 2-(8-oxo-7-((5-trifluoromethyl)-1H-benzo[d]imidazol-2-yl)methyl)8-dihydropyrazin[(2,3-d]pyridazin-5-yl) acetic acid (formula II), is disclosed in WO 2012/009553 A1. Zopolrestat (formula III) is disclosed in U.S. Pat. No. 4,939,140. Each of the patents, applications, and other references referred to herein are incorporated by reference. The diabetic complications include neuropathy, nephropathy, retinopathy, cataracts and cardiovascular complications, including myocardial infarction and cardiomyopathy. This invention is also directed to combinations of these salts and antihypertensive agents. These combinations are also useful in treating diabetic complications in mammals.

It is well known in the art that highly water soluble medicinal preparations, when administered orally, result in efficient absorption of such preparations from the gastrointestinal tract into systemic circulation. Another hallmark of such preparations is the rapid rate at which they are absorbed into the systemic circulation resulting in a high concentration of the active agent in the blood. Also, water soluble preparations are especially suitable for parenteral administration, for example, intravenous administration.

### SUMMARY OF THE INVENTION

The present invention is directed to water soluble salts of 2-(8-oxo-7((5-trifluoromethyl)-1H-benzo[d]imidazol-2-yl)methyl)8-dihydropyrazin[2,3-d]pyridazin-5-yl)acetic acid (Formula I), of the Formula II, wherein X⁺ is a counter-ion selected from protonated arginine (Formula IIa), lysine (Formula IIb), aspartic acid (Formula IIc), glutamic acid (Formula IId), glucosamine (Formula IIe), meglumine, also known as N-methylglucamine (Formula IIf), and metoformin (Formula IIg).

The present invention is also directed to water soluble salts of zopolrestat of formula IV, wherein Y⁺ is a counter-ion selected from protonated arginine (Formula IVa), lysine (Formula IVb) aspartic acid (Formula IVc), glutamic acid (Formula IVd), glucosamine (Formula IVe), and metformin (IVf).

Accordingly, the compounds of the present invention are each advantageous salt forms of 2-(8-oxo-7-((5-trifluoromethyl)-1H-benzo[d]imidazol-2-yl)methyl)8-dihydropyrazin[2,3-d]pyridazin-5-yl)acetic acid and zopolrestat.

Formula I

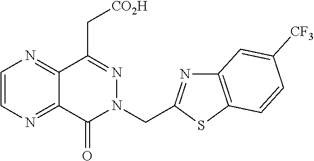 [SMILES: O=C(O)Cc1nn(Cc2nc3cc(C(F)(F)F)ccc3s2)c(=O)c2nccnc12]

Formula II

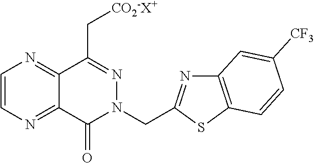

| | X⁺ | | X⁺ |
|---|---|---|---|
| Formula IIa | 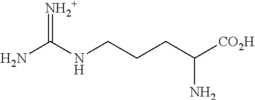 | Formula IIe | 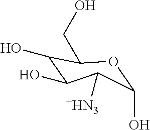 |
| Formula IIb | 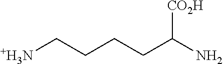 | Formula IIf | 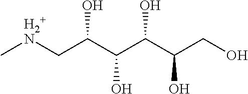 |
| Formula IIc | 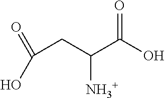 | Formula IIg | 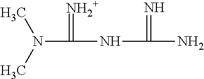 |
| Formula IId | 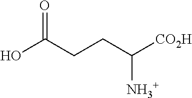 | | |

Formula III

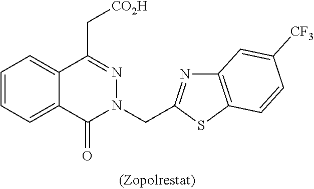 [SMILES: O=C(O)Cc1nn(Cc2nc3cc(C(F)(F)F)ccc3s2)c(=O)c2ccccc12]

Formula IV

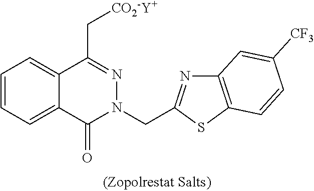

| | Y⁺ | | Y⁺ |
|---|---|---|---|
| Formula IVa | 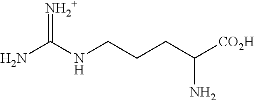 | Formula IVd | 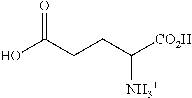 |
| Formual IVb | 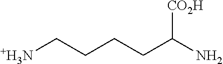 | Formula IVe | 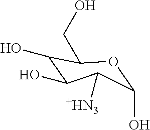 |
| Formula IVc | 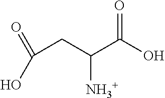 | Formula IVf | 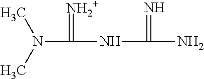 |

The present invention is also directed to pharmaceutical compositions comprising the salts of the present invention and a pharmaceutically acceptable carrier, vehicle or diluent.

The present invention is also directed to a pharmaceutical composition comprising the salts of the present invention and antihypertensive agents. It is preferred that the antihypertensive agents are ACE (angiotensin converting enzyme) inhibitors and/or ARBs (angiotensin receptor blockers). Particularly preferred ACE inhibitor for use in this invention are enalapril, lisinopril, captopril and ramipril. A particularly preferred ARB is losartan.

The present invention is further directed to a method of treating diabetic complications in mammals comprising administering to mammals an effective amount of the salts or compositions to treat diabetic complications. Diabetic complications which are treated by the salts of the present invention and the combinations of this invention include, but are not limited to diabetic neuropathy, diabetic nephropathy, diabetic cardiovascular disease including myocardial infarction, reperfusion injury to the heart tissue and cardiomyopathy.

The present invention is also directed to a method of treating non-diabetic complications in mammals, such as tissue ischemia, including cardiac ischemia, myocardial infarction and reperfusion injury to the heart tissue. This invention is also directed to combinations of the salts of the present invention with antihypertensive agents for said non-diabetic complications in mammals.

The present invention is also directed to a kit comprising:
a) a first unit dosage form comprising salts of the Formula II, wherein X⁺ is represented by the Formula IIa-g and a pharmaceutically acceptable carrier, vehicle or diluent;
b) a second unit dosage form comprising a second pharmaceutical agent, a prodrug thereof or a pharmaceutically acceptable salt of the second pharmaceutical agent or the prodrug and a pharmaceutically acceptable carrier, vehicle or diluent; and
c) a container.

The present invention is also directed to a kit comprising:
a) a first unit dosage from comprising salts of the present invention of the formula IV, wherein Y⁺ is represented by the formula IVa-f and a pharmaceutically acceptable carrier, vehicle or diluent;
b) a second unit dosage from comprising a second pharmaceutical agent, a prodrug thereof or a pharmaceutically acceptable salt of the second pharmaceutical agent or the prodrug and a pharmaceutically acceptable carrier, vehicle or diluent; and
c) a container.

The present invention is particularly directed to such a kit wherein said second pharmaceutical agent is a ACE inhibitor or ARB.

The term “treating”, “treat” or “treatment” as used herein includes curative, preventative (e.g., prophylactic) and palliative treatment.

Delivery of the aldose reductase inhibitor salts of 2-(8-oxo-7-((5-trifluoromethyl)-1H-benzo[d]imidazol-2-yl)methyl)7,8-dihydropyrazin[2,3-d]pyridazin-5-yl) acetic acid and zopolrestat via the intravenous route is central to treat patients with cardiovascular complications in an emergency setting, for example, patients who are brought in to emergency hospital care. It is important that the salt counter-ion is pharmaceutically acceptable as well as safe for use. The counter-ion (cation) in all the salts of the present invention, except the ones derived from metformin cation belong to Class I counter-ion, as listed in “Handbook of Pharmaceutical Salts Properties, Selection, and Use,” P. Heinrich Stahl, Camille G, Wermuth (eds), Second Revised Edition. Metformin has been in human therapeutic use for over three decades and it is the largest selling drug in the world, by weight. Also, it is one of the safest drugs.

Mylari, in U.S. Pat. No. 6,570,013 B2, has disclosed zopolrestat salts with protonated ethanolamine, diethanolamine and triethaolamine as counter ions.

Johnson, in U.S. Pat. No. 5,990,111, has described the clinical utility of zopolrestat in preventing or reversing diabetic cardiomyopathy in diabetic patients.

It should be understood that the location of the positive charge in metformin is illustrative only and it could be located on other nitrogen atoms in metformin.

### DETAILED DESCRIPTION OF THE INVENTION

The salts of the present invention are highly water soluble forms of 2-(8-oxo-7-((5-trifluoromethyl)-1H-benzo[d]imidazol-2-yl)methyl)8-dihydropyrazin[2,3-d]pyridazin-5-yl)acetic acid and zopolrestat. Accordingly, these compounds are each advantageous salt forms of 2-(8-oxo-7-((5-trifluoromethyl)-1H-benzo[d]imidazol-2-yl)methyl)8-dihydropyrazin[2,3-d]pyridazin-5-yl)acetic acid and zopoltestat.

The salts of the present invention, i.e., 2-(8-oxo-7-((5-trifluoromethyl)-1H-benzo[d]imidazol-2-yl)methyl)8-dihydropyrazin[2,3-d]pyridazin-5-yl)acetic acid arginine salt (IIa), 2-(-8-oxo-7-((5-trifluoromethyl)-1H-benzo[d]imidazol-2-yl)methyl)8-dihydropyrazin[2,3-d]pyridazin-5-yl)acetic acid lysine salt (IIb), 2-(8-oxo-7-((5-trifluoromethyl)-1H-benzo[d]imidazol-2-yl) methyl)8-dihydropyrazin[2,3-d]pyridazin-5-yl)acetic acid aspartic acid salt (IIc), 2-(8-oxo-7-((5-trifluoromethyl)-1H-benzo[d]imidazol-2-yl)methyl)8-dihydropyrazin[2,3-d]pyridazin-5-yl)acetic acid glutamic acid salt (IId), 2-(8-oxo-7-((5-trifluoromethyl)-1H-benzo[d]imidazol-2-yl)methyl)8-dihydropyrazin[2,3-d]pyridazin-5-yl)acetic acid glucosamine salt (IIe), 2-(8-oxo-7-((5-trifluoromethyl)-1H-benzo[d]imidazol-2-yl)methyl)8-dihydropyrazin[2,3-d]pyridazin-5-yl)acetic acid glucamine salt (IIf), 2-(8-oxo-7-((5-trifluoromethyl)-1H-benzo[d]imidazol-2-yl)methyl) 8-dihydropyrazin[2,3-d]pyridazin-5-yl)acetic acid metformin salt (IIg), and zopolrestat arginine salt (IVa), zopolrestat lysine salt (IVb), zopolrestat aspartic acid salt (IVc), zopolrestat glutamic acid salt (IVd), zopolrestat glucosamine salt (IVe) and zopolrestat metformin salt (IVf) are readily prepared as set forth below. 2-(8-oxo-7-((5-trifluoromethyl)-1H-benzo[d]imidazol-2-yl)methyl)8-dihydropyrazin[2,3-d]pyridazin-5-yl)acetic acid or zopolrestat is dissolved in an appropriate reaction inert solvent. As used herein the expression “reaction inert solvent” refers to a solvent or a mixture of solvents which does not interact with starting materials, reagents, intermediates or products in a manner which adversely affects the yield of the desired product. Alcohol solvents and ketone solvents can be used. Preferred solvents include methanol, ethanol, isopropanol, acetone, ethyl methyl ketone, diethyl ketone and methyl isobutyl ketone. The reaction mixture is stirred at about 0° C. to about the refluxing temperature of the solvent being used for about 2 to 4 hours. The salt of this invention is isolated from the reaction mixture by methods well known to those skilled in the art. It is preferred that the reaction mixture is directly evaporated. The residue from the evaporation is preferably crystallized from an appropriate solvent or mixture of solvents.

2-(8-oxo-7-((5-trifluoromethyl)-1H-benzo[d]imidazol-yl)methyl)8-dihydropyrazin[2,3-d]pyridazin-5-yl)acetic acid is prepared as disclosed in WO 2012/009553 A1, which is incorporated herein by reference. Zopolrestat is prepared as disclosed in U.S. Pat. No. 4,939,140.

Measurement of the water solubility of the salts of the present invention is accomplished by using methods well known to those skilled in the art. Specifically, to a weighed amount of the salt of the present invention distilled water is added in small portions until a clear solution is obtained. The total volume of the solution is measured. The water solubility of the particular salt, in mg/mL, is calculated by dividing the weight of the salt, in mg, by the volume of the solution, in mL.

ACE inhibitors which are within the scope of this invention include, but are not limited to: benazepril, which may be prepared as disclosed in U.S. Pat. No. 4,410,520; captopril, which may be prepared as disclosed in U.S. Pat. Nos. 4,046,889 and 4,105,776; enalapril, which may be prepared as disclosed in U.S. Pat. No. 4,374,829; lisinopril, which may be prepared as disclosed in U.S. Pat. No. 4,555,502: and ramapril which may be prepared as disclosed in U.S. Pat. No. 4,587,258.

Angiotensin-II receptor blockers (ARBs, also known as angotensin-II receptor antagonists) which are within the scope of this invention include, but are not limited to, losartan, which may be prepared as disclosed in U.S. Pat. No. 5,138,069; valsartan, which may be prepared as disclosed in U.S. Pat. No. 5,399,578; and irbesartan, which may be prepared as disclosed in U.S. Pat. No. 5,270,317.

Generally, an active composition of this invention is administered orally, or parenterally (e.g., intravenous, intramuscular, subcutaneous or intratmedullary). Topical administration may also be indicated, for example, where the patient is suffering from gastrointestinal disorders or whenever the medication is best applied to the surface of a tissue or organ as determined by the attending physician.

For intravenous use, the salts of the present invention can be administered by preparing the salts in situ.

For buccal administration the active composition of this invention (two active agents administered together or separately) may take the form of tablets or lozenges formulated in a conventional manner.

For intranasal administration or administration by inhalation, the active compositions of the invention (two active agents administered together or separately) are conveniently delivered in the form of a solution or suspension from a pump spray container that is squeezed or pumped by the patient or as an aerosol spray presentation from a pressurized container or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. The pressurized container or nebulizer may contain a solution or suspension of the active compound or combination of compounds. Capsules and cartridges (made, for example, from gelatin) for use in an inhaler or insufflator may be formulated containing a powder mix of a compound or compounds of the invention and a suitable powder base such as lactose or starch.

For purposes of transdermal (e.g., topical) administration, dilute sterile, aqueous or partially aqueous solutions (usually in about 0.1 % to 5% concentration), otherwise similar to the above parenteral solutions, are prepared.

Methods of preparing various pharmaceutical compositions with a certain amount of active ingredient are known, or will be apparent in light of this disclosure, to those skilled in this art. For examples of methods of preparing pharmaceutical compositions, see Remington's Pharmaceutical Sciences, Mack Publishing Company, Easton, Pa. 19^{th} Edition (1995), which is incorporated herein by reference.

The active compositions of this invention contain an amount of both salts of the present invention or an amount of one of the salts of the present invention and a second pharmaceutical agent. The amount of each of those ingredients may independently be, for example, 0.0001%-95% of the total amount or the composition, where the total amount may not, of course, exceed 100%.

The kit form is particularly advantageous when the separate components are preferably administered in different dosage forms (e.g., oral and parenteral), are administered at different dosage intervals, or when titration of the individual components of the combination is desired by the prescribing physician.

The following examples are meant to be illustrative but not limited of the scope of the invention.

### EXAMPLE 1

### Diabetic Rat Model

The following example describes a diabetic rat model that may be used for determination of conditions leading to a method for treatment and prevention of post-ischemic damage of the heart and heart tissue.

Spontaneously diabetic Bio-Bred (BB/W) rats from the colony maintained at the University of Massachusetts Medical Center, Worcester, Mass. were used in this study. BB/W rats were chosen for the current study because BB/W rats have been considered a useful model of autoimmune human insulin-dependent diabetes mellitus (IDDM). Like human IDDM, spontaneous diabetes appears during adolescence, with an abrupt clinical onset characterized by weight loss, hyperglycemia, hypoinsulinemia, and ketonuria. As in the case of human diabetics, pathological changes in retina, myocardium, liver, kidney, bone metabolism and peripheral nerves have all been well documented in BB rats, as described in Diab. Metab. Rev., 8:9 (1992). The BB/W rats were 3 to 4 months old and weighed about 300 to 350 g. The BB/W rats received daily insulin, which was discontinued 24 h prior to performing the isolated heart perfusion studies, leading to a hyperglycemic state. The rats were acutely diabetic, receiving 2.02±0.04 units of insulin daily, and had been diabetic for at least 12±3 days. The mean blood glucose levels in these diabetic rats were 386±24 mg/dL. The age-matched non-diabetic controls had mean blood glucose levels of 92±12 mg/dL.

### EXAMPLE II

### Isolated Perfused Heart Model

This example describes an isolated perfused rat heart model used in development of the invention. Studies are performed using an isovolumic isolated rat heart preparation. Acutely diabetic male BB/W rats and non-diabetic age-matched (3 to 4 months old) control are pretreated with heparin (1000 u; IP), followed by sodium pentobarbital (65 mg/kg; IP). After deep anesthesia is achieved, as determined by the absence of a foot reflex, the hearts are rapidly excised and placed into iced saline. The arrested hearts are retrograde perfused in a non-recirculating model through the aorta within 2 minutes following their excision. Left ventricular developed pressure (LVDP) is determined using a latex balloon in the left ventricle with high pressure tubing connected to a pressure transducer. Perfusion pressure is monitored using high pressure tubing off the perfusion line. Hemodynamic measurements are recorded on a 4-channel Gould recorder. The system has two parallel perfusion lines with separate oxygenators, pumps and bubble traps, but common temperature control allows rapid change perfusion media. The hearts are perfused using an accurate roller pump. The perfusate consists of 118 mM NaCl, 0.47 mM KCl, 12 mM CaCl₂, 12 mM MgCl2, 25 mM NaHCO₃, and the substrate 11 mM glucose. The perfusion apparatus is tightly temperature-controlled, with heated baths being used for the perfusate and for the water jacketing around the perfusion tubing to maintain heart temperature at 37±0.5° C. under all conditions. The oxygenated perfusate in the room temperature reservoir is passed through 25 ft. of thin-walled silicone tubing surrounded by distilled water at 37° C. saturated with 95% oxygen.

The perfusate then enters the water-jacketed (37° C.) tubing leading to the heart through a water jacketed bubble trap. This preparation provides excellent oxygenation that routinely has been stable for 3 to 4 hours.

### EXAMPLE II

### Model for Zero-/Low Ischemia

This example describes a procedure used for study of zero-flow ischemia in diabetic control, diabetic treated, non-diabetic treated and control isolated hearts. Diabetic control (DC), diabetic treated (DZ), normal (C) control, and normal treated (CZ) hearts are subjected to 20 minutes of normoxic perfusion followed by 20 minutes of zero-flow ischemia where the perfusate flow is completely shut off, followed by 60 minutes of reperfusion. Hearts are treated with 10 μM salts of the present invention represented in formula II and IV. In the present examples compounds of the Formula II treated diabetic group (DZ), hearts are subjected to 10 minutes of normoxic perfusion with normal Krebs-Henseleit buffer and 10 minutes of normoxic perfusion with Krebs-Henseleit buffer containing 10 μM present compounds of Formula II. The hearts are then subjected to 20 minutes of zero-flow ischemia followed by 60 minutes of reperfusion. In order to avoid any variability in reperfusion conditions, both DC and DZ hearts are reperfused with normal Krebs-Henseleit buffer.

### EXAMPLE IV

### Model for Low-Flow Ischemia

This example describes a procedure used for study of low-flow ischemia in diabetic controls, diabetic treated, non-diabetic treated and non-diabetic control isolated hearts. Diabetic control hearts (DC) are subjected to 20 minutes of normoxic perfusion at a flow rate of 12.5 mL/minute followed by 30 minutes of low-flow ischemia where the perfusate flow is slowed down to 1.25 mL/min, that is about 10% of normal perfusion, followed by 30 minutes of reperfusion at a normal flow rate (12.5 mL/min). In the salts of the present invention represented in formula II and IV treated diabetic or non-diabetic groups (DZ or CZ), hearts are subjected to 10 minutes of normoxic perfusion (flow rate 12.5 mL/min) with normal Krebs-Henseleit buffer and 10 minutes of normoxic perfusion with Krebs-Henseleit buffer containing 10 μM present compounds metformin the formula II. The hearts are subjected to 30 minutes of low-flow ischemia (flow rate 1.25 mL/min) and 30 minutes of reperfusion at normal flow rate (12.5 mL/min).

Animal models to determine the effects of compounds of the invention on diabetes and complications of diabetes have been reviewed by Tirabassi et al., ILAR Journal, 2004, 45, 292-302. Antidiabetic activity may also be tested according to protocols described in the following patents: U.S. Pat. Nos. 4,340,605; 4,342,771; 4,367,234; 4,617,312; 4,687,777 and 4,703,052. Additional references relevant to this application include the following: French Patent 2796551 and United States Published Patent Application No. 2003/0220301.

### EXAMPLE V

### Synthesis of Compounds

### EXAMPLE VA

One equivalent of 2-(8-exo-7-((5-trifluoromethyl)-1H-benzo[d]imidazol-2-yl)methyl) 8-dihydropyrazin[2,3-d]pyridazin-5-yl)acetic acid or zopolrestat may be dissolved in an appropriate reaction inert solvent. The solvent may be a polar solvent such as water. As used herein, the expression “reaction inert solvent” refers to a solvent or a mixture of solvents which doesn't interact with starting materials, reagents, intermediates or products in a manner which adversely affects the yield of the desired product. Preferred solvents include methanol, ethanol, n-propanol, isopropanol, acetone, acetonitrile ethyl methyl ketone, diethyl ketone, and methyl isobutyl ketone. Particularly preferred solvents for this reaction are acetone, acetonitrile and methanol. To this solution may be added a solution of one equivalent of amine compounds (arginine, lysine, aspartic acid, glutamic acid, glucosamine, meglumine and metformin for Formula II compounds and arginine, lysine, aspartic acid, glutamic acid, glucosamine and metformin for Formula IV compounds). (All of these starting material amine compounds are commercially available.) The resulting reaction mixture can be stirred at about ambient temperature to about the reflux temperature of the solvent being used for about ½ hour to about six hours, preferably at ambient temperature for about two hours. The salts of the present invention, as shown in Formula II, and Formula IV can be isolated from the reaction mixture by methods well known to those skilled in the art, including freeze drying to remove the excess solvents and according to the method of U.S. Pat. No. 6,570,013 B2

### EXAMPLE VB

One equivalent of 2-(8-oxo7-((5-trifluoromethyl)-1H-benzo[d]imidazol-2-yl)methyl)8-dihydropyrazin[2,3-d]pyridazin-5-yl)acetic acid or zopolrestat may be dissolved in an appropriate reaction inert solvent. The solvent may be a polar solvent such as water. As used herein, the expression “reaction inert solvent” refers to a solvent or a mixture of solvents that do not interact with starting materials, reagents, intermediates or products in a manner which adversely affects the yield of the desired product. Preferred solvents include methanol, ethanol, n-propanol, isopropanol, acetone, acetonitrile ethyl methyl ketone, diethyl ketone and methyl isobutyl ketone. Particularly preferred solvents for this reaction are acetone, acetonitrile and methanol. To this solution may be added a solution of more than one equivalent of amine compounds (arginine, lysine, aspartic acid, glutamic acid, glucosamine, meglumine and metformin for Formula II compounds and arginine, lysine, aspartic acid, glutamic acid, glucosamine and metformin for Formula IV compounds. The reaction mixture can be stirred at about ambient temperature to about the reflux temperature of the solvent being used for about 1/2 hour to about six hours, preferably at ambient temperature for about two hours. The salts of the present invention, as shown in Formula II, and Formula IV can be isolated from the reaction mixture by freeze drying, for example, to remove the excess solvent. The resulting freeze dried residues can be used directly to formulate sterile solutions for parenteral administration, e.g., intravenous administration.

What is claimed is:

1. A compound of Formula II,
Formula II

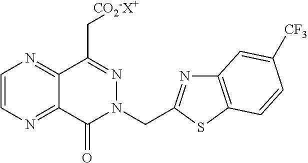

| | X⁺ |
|---|---|
| Formula IIa | 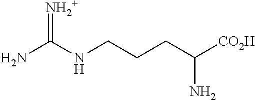 |
| Formula IIb | 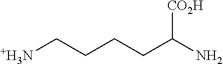 |
| Formula IIc | 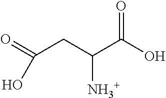 |
| Formula IId | 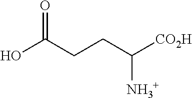 |
| Formula IIe | 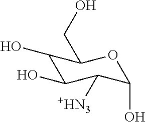 |
| Formula IIf | 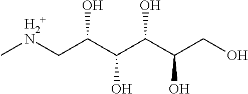 |
| Formula IIg | 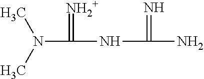 |

wherein X⁺ is protonated arginine (Formula IIa), lysine (Formula IIb), aspartic acid (Formula IIc), glutamic acid (Formula IId), glucosamine (Formula IIe), glucamine (Formula IIf), and metformin (Formula IIg).

2. A compound of Formula IV,
Formula IV

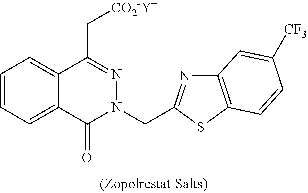

| | Y⁺ |
|---|---|
| Formula IVa | 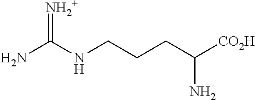 |
| Formula IVb | 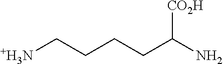 |
| Formula IVc | 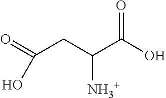 |
| Formula IVd | 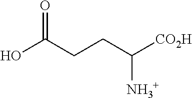 |
| Formula IVe | 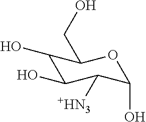 |
| Formula IVf | 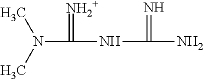 |

wherein Y⁺ is protonated arginine (Formula IVa), lysine (Formula IVb), aspartic acid (Formula IVc), glutamic acid (Formula IVd), glucosamine (Formula IVe), and metformin (Formula IVf).

3. A pharmaceutical composition comprising a compound according to claim 1 and a pharmaceutically acceptable carrier.

4. A pharmaceutical composition comprising a compound according to claim 2 and a pharmaceutically acceptable carrier.

5. A kit comprising a) a unit dosage comprising the compound of claim 1; b) instructions on how to use the kit; and c) at least one container for holding the unit dosage forms.

6. A kit comprising a) a unit dosage comprising the compound of claim 2; b) instructions on how to use the kit; and c) at least one container for holding the unit dosage forms.

7. A method of treating diabetes complications in a mammal, comprising administering to a mammal in need of such treatment a compound according to claim 1.

8. A method of treating diabetes complications in a mammal, comprising administering to a mammal in need of such treatment an anticomplications effective amount of a compound according to claim 1.

9. A method of treating diabetes complications in a mammal, comprising administering to a mammal in need of such treatment an antidiabetic effective amount of a compound according to claim 2.

10. A method of treating diabetes complications in a mammal, comprising administering to a mammal in need of such treatment an antidiabetic effective amount of a compound according to claim 2.

11. A method of treating non diabetic tissue ischemia and cardiovascular diseases, including myocardial infarction and reperfusion injury to the heart tissue in a mammal, comprising administering to a mammal in need of such treatment a compound according to claim 1.

12. A method of treating non diabetic tissue ischemia and cardiovascular diseases, including myocardial infarction and reperfusion injury to the heart tissue in a mammal, comprising administering to a mammal in need of such treatment a compound according to claim 2.

13. The method according to claim 7 wherein the diabetic complications include diabetic neuropoathy, diabetic nephropathy, diabetic retinopathy, cataracts, and diabetic cardiovascular diseases, including myocardial infarction, reperfusion injury to the heart tissue and cardiomyopathy.

14. The method according to claim 9 wherein the said diabetic complications include diabetic neuropoathy, diabetic nephropathy, diabetic retinopathy, cataracts, and diabetic cardiovascular diseases, including myocardial infarction, reperfusion injury to the heart tissue and cardiomyopathy.
